(19) United States
(12) Patent Application Publication
(10) Pub. No.: US 2004/0030211 A1
(43) Pub. Date: Feb. 12, 2004

(54) Maximizing meta-isomers in the production of dialkylbenzene compounds

(76) Inventors: Chung-Ming Chi, Weymouth, MA (US); Shiou-Shan Chen, Winchester, MA (US)

Correspondence Address: EXXONMOBIL CHEMICAL COMPANY, P O BOX 2149, BAYTOWN, TX, 77522-2149, US

(21) Appl. No.: 10/363,515

(22) PCT Filed: Sep. 25, 2001
(86) PCT No.: PCT/US01/29956

(30) Foreign Application Priority Data
Sep. 25, 2000 (US) 60235176

(51) Int. Cl. C07C 2/68

(52) U.S. Cl. 585/467

(57) ABSTRACT

A process for preparing a mixed dialkylbenzene product is disclosed in which a predominant proportion above 60 wt. % of the meta-dialkylbenzene isomer and a very low proportion of the ortho-dialkylbenzene isomer is produced by a liquid-phase alkylation of a suitable olefin and aromatic feed, utilizing an alkylation catalyst selected for enhancement of meta-isomer formation followed by or carried out in combination with a meta-isomer enhancement utilizing a catalyst selected for enhancement of meta-isomer formation.

The present invention relates generally to improvements in liquid phase alkylation and transalkylation processes for the production of dialkylbenzene compounds, particularly diisopropylbenzene (DIPB), from an aromatic feedstock in order to produce predominant proportions of meta-isomers while simultaneously minimizing to very low levels the proportions of ortho-isomers.

### BACKGROUND OF THE INVENTION

Alkylation of aromatic hydrocarbons, such as benzene, with olefins, such as ethylene and propylene, in the presence of suitable catalysts is well known in the art as a way to produce many different alkylated aromatic products. A variety of materials, particularly those generally known as zeolites, have been found useful as alkylation catalysts for these reactions. In general, the zeolite catalysts comprise crystalline metallic aluminosilicates. Zeolites come in many forms having differing properties as catalysts. These alkylation reactions can be carried out in single or multi-bed reactors, and operating parameters can be varied according to the desired alkylation products.

One familiar and useful application of such catalyzed alkylation reactions is in the production of dialkylbenzene products, for example diisopropylbenzene (DIPB). Dialkylbenzene compounds can exist in three isomeric forms, for example DIPB can exist as para-DIPB (PDIPB), meta-DIPB (MDIPB), and ortho-DIPB (ODIPB). The different isomers of dialkylbenzene products typically have different commercial applications, for example as starting materials for the synthesis of other organic compounds. In conventional alkylation reactions, mixtures of the para-, meta-, and ortho-isomers of dialkylbenzenes are produced, and these isomers must be separated and purified by downstream processes in order to be useful for applications which are isomer-specific. Typically such isomer separation processes are difficult and expensive. Because the three isomers generally are close in boiling points, relatively simple and inexpensive fractionation will not ordinarily yield very high purity isomer products. For example, the boiling points at 1 atm (atmospheric pressure) for para-, meta-, and ortho-DIPB respectively are about 210.5° C., 203.2° C. and 204° C. Because of the especially close boiling points of meta-DIPB and ortho-DIPB, this separation is particularly difficult and expensive to carry out. Separation of isomers by crystallization techniques is limited by eutectic points. Thus, it is desirable to design dialkylbenzene production processes, if possible, so as to favor the more desired isomer thereby minimizing separation costs.

In DIPB production, para-DIPB has conventionally been the favored isomer. Thus, U.S. Pat. No. 3,763,259 (Hervert), which patent is incorporated herein by reference, teaches an alkylation process expressly directed to producing para-DIPB. In the Hervert '259 patent, para-DIPB is produced by alkylating benzene with propylene followed by steps for separating unalkylated benzene, separating cumene, separating polyisopropylbenzenes, and separating ortho-DIPB and meta-DIPB from the alkylation reaction product stream to provide a para-DIPB product. The unalkylated benzene is recycled to the alkylation step; cumene and polyisopropylbenzenes are combined and disproportionated; and, the disproportionated hydrocarbons are combined with the ortho-DIPB and meta-DIPB isomers, and the mixture is subjected to an isomerization step. The isomerized hydrocarbons are recycled to the benzene separation step.

A Japanese patent document JP 56133224 teaches a process to obtain a dialkylbenzene isomeric mixture with a high p-isomer content selectively, by alkylating a monoalkylbenzene with an olefin, etc. in the presence of a specific acid extraction mordenite zeolite exchanged with hydrogen as a catalyst. More particularly, a monoalkylbenzene, particularly toluene or cumene, is reacted with an alkylating agent selected from an olefin, an alcohol and an alkyl halide in the vapor phase in the presence of a catalyst consisting of (A) an acid extraction mordenite zeolite exchanged with hydrogen ions, prepared by treating a mordenite zeolite with an acid, and having a SiO₂/Al₂O₃ ratio of 15-100 and an Na ion content<=0.5 wt. %, or (B) the zeolite exchanged with a metallic ion or metallic oxide other than alkali metals and/or impregnated with a metallic oxide, preferably at 250-500° C., in the vapor phase to give the titled mixture with a high p-isomeric content.

A more recent patent, U.S. Pat. No. 4,822,943 (Burress), which patent is also incorporated herein by reference, is entitled “Production of Para-diisopropylbenzene,” and teaches a liquid- or vapor-phase alkylation process utilizing a specific alkylation catalyst to favor production of para-DIPB. Specifically, this patent teaches a process wherein cumene and/or benzene is brought into contact with propylene in the presence of a zeolite-type catalyst identified as ZSM-12 to selectively produce para-DIPB. The many examples in the Burress '943 patent show that by varying the choice of liquid- or vapor-phase, alkylation catalyst, liquid-phase or vapor-phase alkylation conditions, alkylation reactor temperature, and other process parameters, the ratios of para-DIPB to meta-DIPB produced could be varied from as high as about 86% para/14% meta (Table 5) to as low as about 33% para/67% meta (Example 2, Table 2), while maintaining ortho-DIPB at low levels of about 1% or less. Based on the Burress '943 data, the highest proportion of meta-isomer attainable using liquid-phase alkylation is less than 50%.

U.S. Pat. No. 4,447,666 (McWilliams), which is also incorporated herein by reference, teaches a method for preparing a para-selective zeolite-based aromatics alkylation catalyst by treating a ZSM-5 zeolite base catalyst. Catalysts prepared according to the McWilliams '666 patent were found to provide alkylated aromatic product mixtures having exceptionally high proportions of the para-dialkylbenzene isomer. U.S. Pat. No. 4,613,717 (Ishida), which is also incorporated herein by reference, similarly teaches a method for preparing an alkylation catalyst useful in vapor phase reaction of benzene or a monoalkylbenzene with an alkylating agent for preferential production of 1,4-dialkyl-benzenes. Ishida '717 teaches that use of the specific Ishida-type catalyst results in both high yield and high selectivity, and is an improvement over the ZSM-5 catalyst taught by the prior art.

U.S. Pat. No. 4,347,393 (Miki), which is also incorporated herein by reference, teaches a process for co-production of cumene and DIPB from starting feeds of benzene and propylene utilizing in combination an alkylation step, a transalkylation step, a catalyst separation step, a neutralization step, and a fractional distillation step. U.S. Pat. No. 4,547,604 (Olah), which is also incorporated herein by reference, teaches a process for the selective isopropylation of cumene to meta-DIPB utilizing a superacid environment as a preliminary step in the manufacture of resorcinol. The Olah '604 patent teaches that the high purity (98-100%) meta-DIPB, substantially free of other DIPB isomers, needed for resorcinol production can be obtained by treating any mixture of DIPB isomers with an excess of anhydrous hydrogen fluoride, or with a perfluorinated alkanesulfonic acid of one to eighteen carbon atoms and a Lewis acid fluoride, or by alkylating or transalkylating cumene with a propyl alkylating agent in one of these superacid systems.

Significantly, the Olah '604 patent notes the difficulties experienced with previous approaches to producing meta-DIPB, observing (col. 3, lines 10-13) that conventional methods for the preparation of meta-DIPB “consist of isopropylation of cumene, giving isomeric mixtures with the ortho and para isomers predominating. Subsequent acid catalyzed isomerization, using usual Friedel-Crafts conditions or solid acid catalysts, increase the amount of meta-isomer up to about 60% of the mixture, but still necessitate separation of the isomers which, due to their close boiling ranges, can generally not be effected by simple distillation alone and thus separation of the isomers is difficult to achieve and expensive.” On the other hand, the process of Olah '604 requires the use of special equipment to accommodate the highly corrosive and difficult to handle superacidic environments, particularly when using hydrogen fluoride and the like, and necessarily includes catalyst separation and neutralization steps requiring still additional equipment and added costs. These drawbacks limit the utility of the Olah '604 process in producing meta-DIPB alone or in coproduction with cumene.

Thus, there remains an unmet need in the art for an efficient process for producing predominant proportions of meta-dialkylbenzene isomer in a catalytic alkylation process, or for co-producing cumene and meta-DIPB, using conventional processing equipment and without the need for highly corrosive reagents and special additional processing steps. The aforementioned drawbacks and limitations of the prior art are overcome, in whole or in part, with the methods of this invention for maximizing the meta-isomers in the production of dialkylbenzene compounds.

### OBJECTS OF THE INVENTION

Accordingly, a principal object of this invention is to provide single or multi-stage methods for alkylating an aromatic compound with an alkylating agent so as to produce a dialkylbenzene product having predominant proportions of the meta-isomer.

It is a general object of this invention to provide an integrated alkylation process that includes an alkylation step utilizing particular categories of alkylation catalyst, preferably an acidic zeolite-based alkylation catalyst, to preferentially produce meta-isomers of dialkylbenzene, in combination with a meta-isomer enhancement step and subsequent separation step to obtain a high purity meta-dialkylbenzene product, the meta-isomer enhancement step either being combined with the alkylation step or being performed downstream from the alkylation step.

A specific object of this invention is to provide an improved liquid-phase alkylation process for maximizing the production of the meta-isomer of a dialkylbenzene while simultaneously minimizing the production of the ortho-isomer to about 1 wt. % or less.

Another specific object of this invention is to provide an improved alkylation process for coproduction of cumene and/or para-DIPB together with a high purity meta-DIPB product.

A further specific object of this invention is to provide a process for producing high purity meta-DIPB as a coproduct of cumene production utilizing alkylation and transalkylation reaction zones in series with high purity meta-DIPB recovered from the transalkylation zone effluent.

Yet another specific object of this invention is to provide a process for producing high purity meta-DIPB product from benzene and propylene feeds to a liquid-phase alkylator, and a mixed benzene-DIPB feed to a downstream transalkylator, while keeping production of ortho-DIPB to very low levels below about 1 wt. %, and recovering meta-DIPB from the transalkylator effluent.

Still a further specific object of this invention is to provide a process for producing high purity meta-DIPB product from cumene and propylene feeds, or from a mixed cumene/benzene feed and a propylene feed, to a liquid-phase alkylator, and a mixed benzene-DIPB feed to a downstream transalkylator, with recovery of meta-DIPB from the transalkylator effluent.

Yet a further specific object of this invention is to provide a process for producing high purity meta-DIPB product from a cumene feed or a mixed cumene/benzene feed and a propylene feed in a single reaction zone designed for simultaneous alkylation and transalkylation under conditions that maximize the meta-DIPB/ortho-DIPB ratio with recovery of meta-DIPB from the effluent from the reaction zone.

Other objects and advantages of the present invention will in part be obvious and will in part appear hereinafter. The invention accordingly comprises, but is not limited to, the methods and related apparatus, involving the several steps and the various components, and the relation and order of one or more such steps and components with respect to each of the others, as exemplified by the following description and the accompanying drawings. Various modifications of and variations on the method and apparatus as herein described will be apparent to those skilled in the art, and all such modifications and variations are considered within the scope of the invention.

### SUMMARY OF THE INVENTION

The present invention relates generally to improvements in alkylation processes for the production from an aromatic feedstock of dialkylbenzene compounds, particularly diisopropylbenzene (DIPB), in order to produce predominant proportions of meta-isomers while simultaneously minimizing to very low levels the proportions of ortho-isomers. More specifically, the methods of this invention generally comprise the following interrelated process steps: (1) an alkylation step utilizing a catalyst selected for enhancement of meta-isomer formation to obtain an alkylation effluent having a predominant proportion (above 50% based on total DIPB content) of meta-isomer; (2) a first product separation step, comprising one or more product separation processes, to separate the product stream from the alkylation step into a crude dialkylbenzene product stream and one or more other component streams; (3) treating the crude dialkylbenzene product stream in a meta-isomer enhancement step utilizing a catalyst selected to preferentially further form or to enhance the proportion of meta-isomer to above 60%, preferably above 64%, for example a range of about 64%-67%, while minimizing formation of ortho-isomer; and (4) a second product separation step, comprising one or more product separation processes, to separate the product stream from the meta-isomer enhancement step into a final high purity meta-isomer product stream, predominantly comprising meta-isomer with a very low proportion of undesired ortho-isomer and a recycle stream comprising para-isomer, unreacted aromatic feedstock, and/or byproducts for recycle to an upstream step.

The meta-isomer enhancement step may variously comprise a transalkylation step, an isomerization step, and/or a disproportionation step under suitable conditions. In one variation of the general process, the alkylation and the meta-isomer enhancement steps can be combined and carried out simultaneously in a single reaction zone, for example in a combination alkylator/transalkylator. In this variation, it will be understood that the first product separation step would be eliminated. For every variation of the general process, however, production of the ortho-isomer must be minimized to less than about 1 wt. % in order to achieve economical production of high purity meta-isomer.

### BRIEF DESCRIPTION OF THE DRAWINGS

FIG. 1 is a process flow diagram schematically illustrating an embodiment of a meta-DIPB process according to the present invention designed for recovering only high purity meta-DIPB.

FIG. 2 is a process flow diagram schematically illustrating an alternative embodiment of a meta-DIPB process according to the present invention which optionally provides for also recovering a para-DIPB product.

FIG. 3 is a process flow diagram schematically illustrating an embodiment of a coproduction cumene/meta-DIPB process according to the present invention for recovering a cumene product together with high purity meta-DIPB.

FIG. 4 is a graph illustrating the variation of two variables, the meta-DIPB/para-DIPB ratio and the ortho-DIPB/meta-DIPB ratio, over a DIPB conversion range of 0 to 1.0.

FIG. 5 is a graph illustrating the variation of two variables, % ortho-DIPB/total DIPB and % ortho-DIPB/meta-DIPB, over ratios of benzene to cumene in the aromatic feed of from 0.0 to 1.1 wt. %.

### DETAILED DESCRIPTION OF PREFERRED EMBODIMENTS

The present invention is generally directed to methods for the alkylation of a monoalkylbenzene, or benzene, or a benzene/monoalkylbenzene mixture with an olefin, to yield a product containing predominant proportions of meta-dialkylbenzene isomer which can then be relatively easily processed to produce a very high purity meta-dialkylbenzene final product. A particular application of the methods of this invention is to produce a high purity meta-DIPB product either alone, or as a coproduct with cumene, or as a coproduct with para-DIPB, or as a coproduct with cumene and para-DIPB. In general, the processes of this invention comprise a liquid-phase alkylation zone operating in combination with a meta-dialkylbenzene isomer enhancement zone which, alternatively, may comprise a transalkylation step, a disproportionation step, an isomerization step, or a combination of these processes. Furthermore, the methods of this invention minimize formation of impurities and undesirable byproducts such as o-dialkylbenzene, indans, and other structural isomers commonly found in dialkylbenzene products.

If the meta-dialkylbenzene isomer enhancement zone comprises an isomerization or transalkylation reactor, that isomerization/transalkylation reactor can be operated under a wide variety of conditions. Temperature can range from 100 to 500° C., preferably 150 to 300° C. Pressures can range from 150 to 2000 psig. A weight hourly space velocity (WHSV—weight of feed/weight of catalyst) based on crude dialkylbenzene ranging from 0.2 to 10.0 hr⁻¹, preferably 0.4 to 4.0 hr⁻¹, is attainable. The benzene to crude dialkylbenzene weight ratio can be in the range of 1.0 to 10.0, preferably from 1.5 to 4.0. The m- and, optionally, the p-dialkylbenzene isomers are then recovered in a downstream separation process, such as distillation or a combination of distillation and crystallization. Residual dialkylbenzene and most of the polyalkylbenzenes are recovered and recycled to the isomerization/transalkylation reactor for production of additional product or makeup monoalkylbenzene.

Olefin contacts monoalkylbenzene or a benzene/monoalkylbenzene mixture in an alkylation reactor. The reactor may be operated under varying conditions: temperatures may range from 60° C. to 400° C., preferably from 120° C. to 300° C.; pressures can range from about 200 to 2000 psig; and WHSV based on olefin can range from about 0.1 to 4.0 hr⁻¹, preferably from 0.2 to 1.0 hr⁻¹, to produce crude dialkylbenzene. The aromatics to olefin molar ratio can be from 1.0 to 100, preferably from 2 to 20.

Both the isomerization/transalkylation and the alkylation reactors preferably use suitable acidic zeolite-based catalysts selected from the group as follows: MCM-22, MCM-36, MCM-49, MCM-56, ITQ-1, ITQ-2, ITQ-3, SSZ-25, PSH3 and ERB-1.

We have determined as part of this invention that the suitable acidic zeolite-based catalysts show surprising superiority in long-term stability and in maximizing the production of meta-isomers, while minimizing production of ortho-isomers, as compared with conventional acidic zeolite-based catalysts such as mordenite and ZSM-12. The suitable acidic zeolite-based catalysts, referenced above, have also shown superior performance for purposes of this invention as compared with familiar solid acid-type catalysts such as silica, alumina, Group IVB oxides, zirconium phosphate, silicophosphoaluminate, antimonophosphoaluminate, etc.

The following U.S. and foreign patents and references, each of which is incorporated herein by reference, teach the preparation and/or use of various acidic zeolite-based catalysts: U.S. Pat. Nos. 6,077,498 (Diaz Cabanas); 6,096,288 (Roth); 6,063,262 (Dhingra); 6,049,018 (Calabro); 5,437,855 (Valyocsik); 5,670,131 (Valyocsik); 5,362,697 (Fung); 5,346,685 (Moini); 5,236,575 (Bennett); 5,068,096 (Valyocsik); 5,104,495 (Chang); 4,981,663 (Rubin); 4,826,667 (Zones); 4,439,409 (Puppe); WO97/19021 (Corma); 4,696,807 (Chu); 4,791,088 (Chu); 5,441,721 (Valyocsik); 4,954,325 (Rubin); 5,043,512 (Chu); 5,488,194 (Beck); 4,439,409 (Puppe) and EP-B-293032. Although some of these patents teach the utility of acidic zeolite-based catalyst materials in alkylation and isomerization processes for hydrocarbon processing, none of these patents recognizes the special utility of the acidic zeolite-based catalysts in the preferential formation of meta-isomers in dialkylbenzene production, while simultaneously minimizing the production of undesired ortho-isomers.

Our tests have shown that one of the conventional zeolite-based catalysts, namely zeolite beta, to a limited extent can also be used in practicing the methods of the present invention. Zeolite beta has been found to perform well initially as the catalyst in the alkylation stage of this invention; however, the performance of zeolite beta in the alkylation stage deteriorates over time which makes it less desirable than the acidic zeolite-based alkylation catalysts for long-term use. By contrast, suitable acidic zeolite-based catalysts, such as MCM-22 and those referenced above, have been found to maintain high and stable performance levels in the alkylation stage of the present invention over extended time periods. An example of typical alkylation stage conditions and alkylator effluent composition utilizing beta zeolite as the alkylation catalyst are as follows: feed: benzene and propylene; conditions: 120-150° C. inlet, adiabatic operation, benzene/propylene molar ratio 3-4, propylene WHSV 0.45-0.55 hr⁻¹, recirculation/feed ratio ˜9-10; the m/o/p-DIPB ratio in the alkylator effluent ranges from about 53 to 62/1.8 to 2.8/36 to 44 respectively.

Zeolite beta may also be used, again with limitations, as the catalyst in the meta-isomer enhancement, e.g., transalkylation, step of this invention. We have found, however, that zeolite beta does not perform as well as certain acidic zeolite-based alkylation catalysts, such as MCM-22 and those referenced above, in maximizing the formation of meta-isomer and in minimizing the formation of ortho-isomer in the transalkylator. In fact, the proportion of ortho-isomer in the transalkylator effluent is about twice as high with zeolite beta catalyst as it is with an acidic zeolite-based alkylation catalyst. An example of typical transalkylator stage conditions and transalkylator effluent composition utilizing beta zeolite as the transalkylation catalyst are as follows: feed: benzene and crude PIPB; conditions: 155-180° C., isothermal once-through operation, benzene/PIPB weight ratio 3-6, PIPB WHSV 0.5-0.9 hr⁻¹; the m/o/p-DIPB ratio in the transalkylator effluent ranges from about 65 to 66/0.55 to 0.8/33 to 34 respectively. Although these numbers are within operable ranges for this invention, certain acidic zeolite-based catalysts, such as MCM-22 and those referenced above, are preferred for use in both the alkylation and transalkylation steps of the present invention.

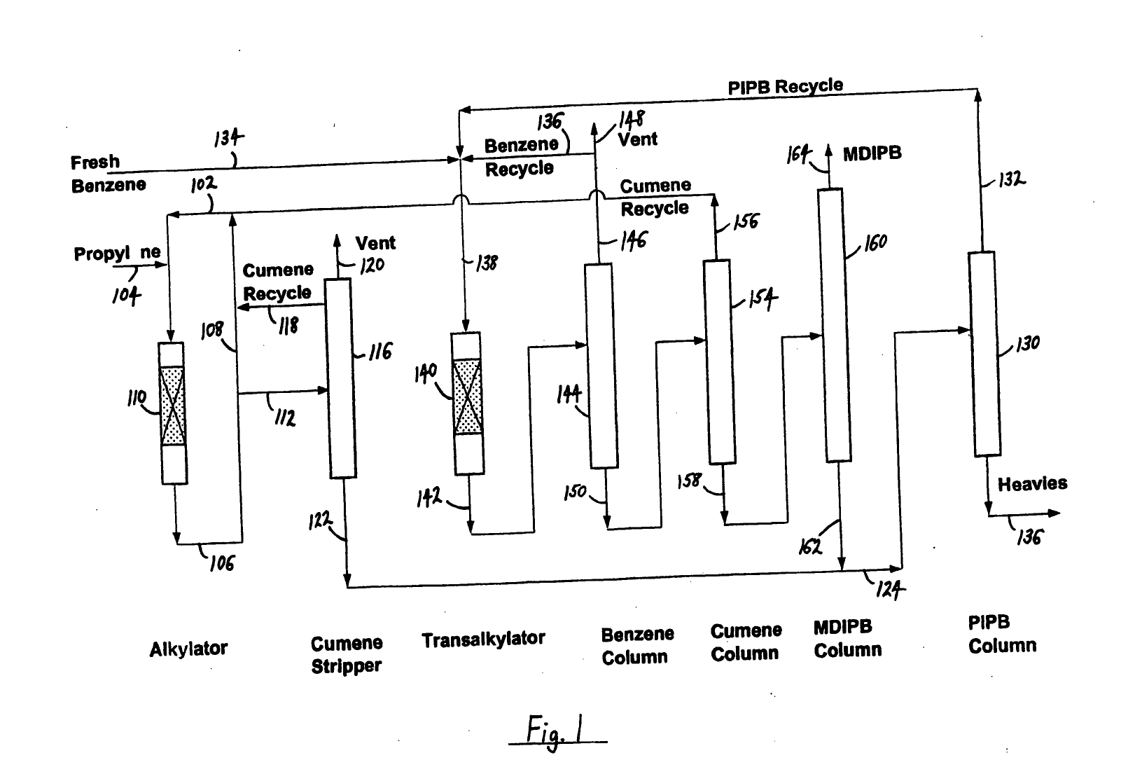

FIG. 1 illustrates a first embodiment of this invention which is directed to recovering only a high purity meta-DIPB product while recycling unreacted aromatic feedstock as well as certain of the aromatic byproducts in order to maximize meta-DIPB recovery. FIG. 1 is shown specifically for the preparation of meta-DIPB, but it is understood that this process flow arrangement could be readily adapted for the preparation of high purity meta-isomers of other dialkylbenzene compounds.

In FIG. 1, cumene or a cumene/benzene mixture (feedstream 102) is brought into contact with a propylene feedstream 104 in an alkylator 110, preferably loaded with an acidic zeolite-based alkylation catalyst, under alkylation conditions. A first alkylation effluent portion (stream 108) of the alkylator effluent stream 106 is recycled to alkylator 110. The remainder (stream 112) of the alkylator effluent stream 106 is sent to a cumene recovery column or stripper 116. A first cumene recycle stream 118 is drawn off an upper region of stripper 116 for mixing with first alkylation effluent portion 108 for recycle to alkylator 110. Volatile alkylation byproducts and residual light components are vented from the top of stripper 116 by vent stream 120. Crude polyisopropylbenzene (comprising DIPB isomers and heavier byproducts such as triisopropylbenzenes) is withdrawn as bottoms stream 122 from stripper 116 and sent to a polyisopropylbenzene recovery section 130.

Polyisopropylbenzene recovery section 130 separates the crude polyisopropyl-benzene product stream 122 into a polyisopropylbenzene recycle stream 132 withdrawn as an overhead stream from recovery column 130, and a relatively small bottoms stream 136 of heavier aromatic components. The polyisopropylbenzene recycle stream 132 is mixed with fresh benzene (benzene feedstream 134) and/or with recycle benzene (benzene recycle stream 136), and the mixed reactant stream 138 is introduced into a meta-isomer enhancement zone. As shown in FIG. 1, the meta-isomer enhancement zone is a transalkylator/isomerizer 140 operating under mild transalkylation conditions using a suitable transalkylation/isomerization catalyst, preferably an acidic zeolite-based suitable for this catalyst.

In the transalkylator/isomerizer 140, a meta-DIPB-rich stream can be produced (due to thermodynamic equilibrium) for later recovery of a high purity meta-DIPB product. The operating conditions in transalkylator/isomerizer 140, particularly temperature, can be controlled to produce enough cumene for subsequent recycle to the alkylator as second cumene recycle stream 156 to supplement first cumene recycle stream 118. Alternatively, in a variation of the process of FIG. 1, cumene recycle stream 118 can be supplemented with a fresh cumene feed (not shown) to alkylator 110. In this alternative process scheme, transalkylator/isomerizer 140 can be operated so as to maximize production of meta-DIPB without regard for meeting upstream cumene requirements.

Effluent stream 142 from the transalkylator/isomerizer 140 is sent to a benzene/cumene recovery section 144. The overhead stream 146 withdrawn from benzene column 144 is separated into the previously-mentioned benzene recycle stream 136 and lighter components which are vented as vent stream 148. The bottoms stream 150 from column 144, comprising cumene and DIPB isomers (predominantly meta-DIPB), is sent to cumene column 154. The overhead stream 156 withdrawn from column 154 is the previously-mentioned cumene recycle stream 156. The bottoms stream 158 from column 154, comprising predominantly meta-DIPB with a very low (typically below about 1 wt. %) proportion of ortho-DIPB and essentially no benzene or cumene, is sent to the meta-DIPB column 160. The bottoms stream 162 from meta-DIPB column 160, comprising mostly para-DIPB, is mixed with the crude polyisopropylbenzene product stream 122 to form a mixed feed 124 going to polyisopropylbenzene recovery section 130. High purity meta-DIPB product is recovered as overhead stream 164 from column 160.

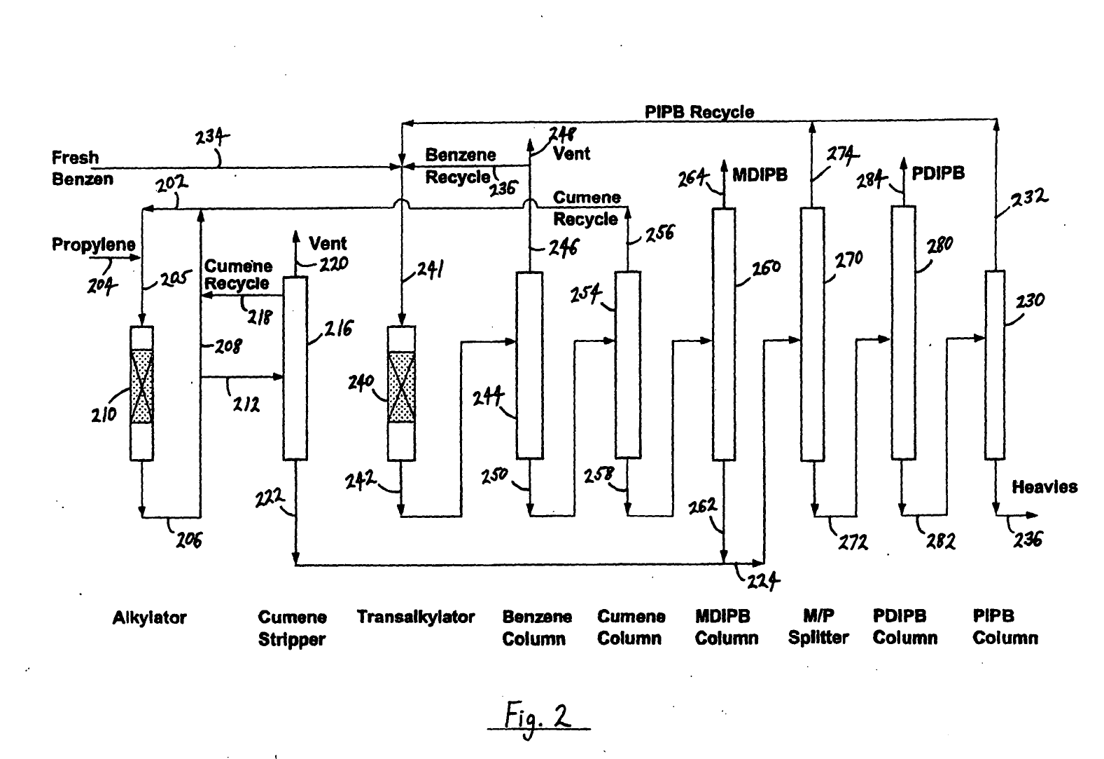

FIG. 2 illustrates a second embodiment of this invention which is directed to an optional variation of the process of FIG. 1 wherein it is desired to also recover a para-DIPB product along with a high purity predominant meta-DIPB product instead of recycling para-DIPB to the transalkylator/isomerizer for at least partial conversion to meta-DIPB. Apparatus components and process streams in FIG. 2 which are the same as or essentially comparable to corresponding apparatus components and process streams in FIG. 1 have the same two last reference numbers but use the “200+” reference numbering series.

The principal differences between the process of FIG. 2 and that of FIG. 1 can readily be seen to be the addition of two additional product separation sections—a meta-isomer/para-isomer splitter column (reference numeral 270) and a para-DIPB recovery column (reference numeral 280). Cumene or cumene/benzene mixture (stream 202) mixes with propylene stream 204 and the combined stream 205 is sent to alkylator 210 operating under alkylation conditions. Part 208 of alkylator effluent 206 recycles back to the alkylator 210. The rest (stream 212) of the alkylator effluent 206 is separated into two parts in a cumene recovery section 216. A cumene stream 218 from column 216 also recycles to the alkylator 210. The crude PIPB stream 222 from column 216 is sent to a MDIPB/PDIPB separation section 270 together with the remaining effluent 262 from the MDIPB recovery section 260. Those molecules lighter than PDIPB are recovered from the top of column 270 as overhead stream 274 and are recycled back to the transalkylator/isomerizer section 240. The PDIPB-rich stream 272, which is almost free of MDIPB, will be sent to a PDIPB recovery section 280 to produce a PDIPB product stream 284. The remaining product stream 282 from the PDIPB recovery section 280 is then sent to a PIPB recovery section 230 to recover any PDIPB left and TIPB (triisopropylbenzenes) as recycle stream 232. Small amounts of the heavy components are also rejected in the PIPB recovery section as bottoms stream 236.

The recovered PIPB streams 232 and 274, respectively from the PIPB recovery column 230 and the MDIPB/PDIPB separation section 270, join with recycle benzene stream 236 and fresh benzene stream 234. Mixed stream 241 enters the transalkylator/isomerizer 240 under transalkylation conditions. In the transalkylator 240, an MDIPB-rich stream 242 can be produced for later recovery of MDIPB product (stream 264). The operating conditions (mainly the temperature) of the transalkylator 240 can be adjusted to produce enough makeup cumene (stream 256) for feeding to alkylator 210. However, if it is desired to maximize DIPB production, it is also possible to import fresh cumene (not shown) as part of the feed 202 to the alkylator 210. The effluent stream 222 from the transalkylator is, in this variation, sent to a benzene/cumene recovery section. The benzene recycle stream 236 will join the PIPB streams 232 and 274 respectively from the PIPB recovery column 230 and the MDIPB/PDIPB separation section 270. The cumene recycle stream 256 will be sent back to the alkylator 210 as makeup. Recovery of high purity MDIPB in stream 264 is achieved by sending the MDIPB-rich but substantially benzene-and-cumene free stream 258 to MDIPB recovery section 260. The PDIPB recovery section 280 in this embodiment may comprise a distillation column or a one or multistage crystallization unit.

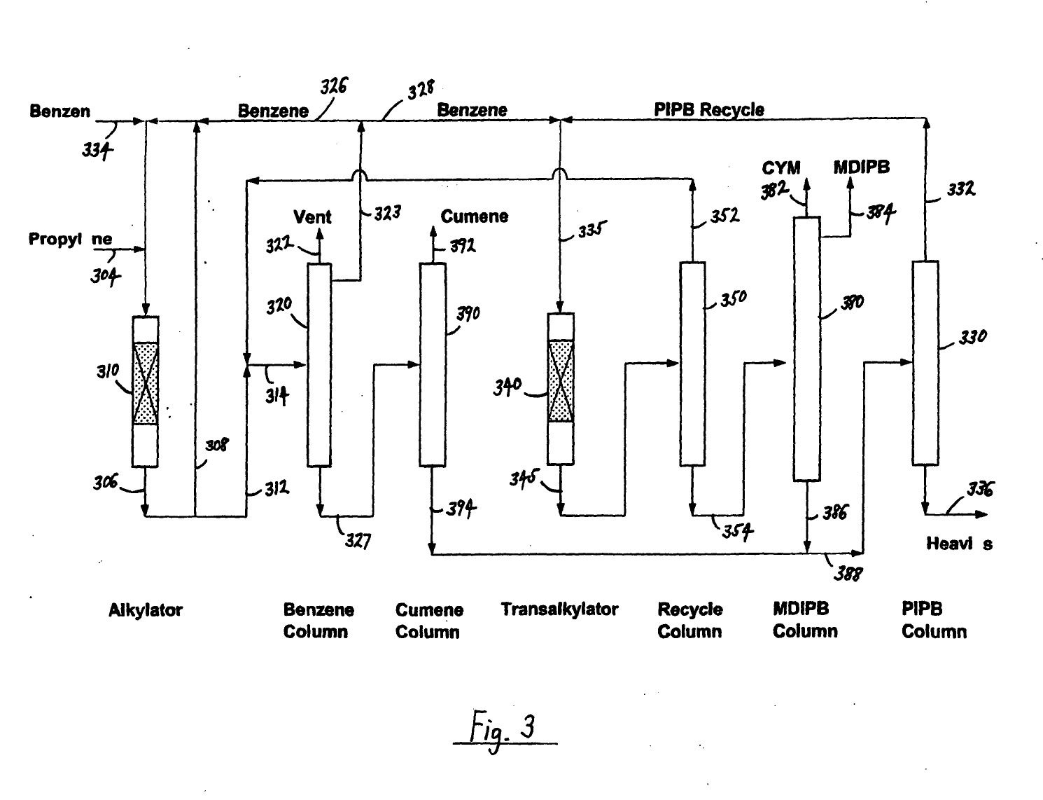

FIG. 3 illustrates a third embodiment of this invention which is directed to an optional variation of the process of FIG. 1 wherein it is desired to also recover a cumene product along with a high purity meta-DIPB product instead of recycling cumene as feed to the alkylator.

In FIG. 3, fresh benzene stream 334 is mixed with recycle benzene stream 326 and brought into contact with a propylene feedstream 304 in an alkylator 310, preferably loaded with an acidic zeolite-based alkylation catalyst under alkylation conditions. A first alkylation effluent portion (stream 308) of the alkylator effluent stream 306 is mixed with recycle benzene stream 326 and recycled to alkylator 310. The remainder (stream 312) of the alkylator effluent stream 306 is mixed with recycle stream 352 from recycle column 350 and sent to a benzene recovery section 320. A benzene stream 323 is drawn as a side stream off an upper region of benzene column 320. A first portion 326 of benzene stream 323 is recycled to alkylator 310. A second portion 328 of benzene stream 323 is mixed with a polyisopropylbenzene recycle stream 332 from the PIPB column 330, and the mixed stream 335 is sent to transalkylator 340. Volatile alkylation byproducts and residual light components are vented from the top of benzene column 320 by overhead vent stream 322.

A bottoms stream 327 comprising crude polyisopropylbenzene, cumene, and heavier byproducts such as triisopropylbenzenes is withdrawn from the bottom of benzene column 320 and sent to a cumene recovery section 390. A cumene product stream 392 is recovered as an overhead stream from column 390. A PIPB stream 394 is withdrawn from the bottom of column 390, mixed with a bottoms stream 386 from the MDIPB column 380, and the mixed stream 388 is sent to a PIPB recovery section which, as shown in FIG. 3, comprises a PIPB column 330. Heavy compounds are removed in bottoms stream 336 from the PIPB column 330. A PIPB recycle stream 332 is withdrawn as an overhead stream from column 330, mixed with portion 328 of the recycle benzene, and the mixed stream 335 is sent to transalkylator 340.

In the transalkylator 340, a meta-DIPB-rich stream can be produced for subsequent recovery of a high purity meta-DIPB product. Effluent stream 345 from transalkylator 340 is sent to benzene/cumene combined recovery section 350. A combined benzene/cumene recycle stream 352 is withdrawn as an overhead stream from column 350 and sent upstream where it is mixed with portion 312 of the alkylator effluent stream 306 and recycled to benzene column 320. A substantially benzene-free and cumene-free meta-DIPB stream is withdrawn from recycle column 350 as bottoms stream 354 and sent to the MDIPB recovery section 380. A high purity meta-DIPB product is recovered as either an overhead stream, or as shown in FIG. 3, a sidedraw stream 384 from an upper region of column 380. As shown in FIG. 3, an overhead stream 382 from column 380 can be used to withdraw cymene byproducts from the system.

A bottoms stream 386 from column 380 is mixed with stream 394 from the cumene column, and combined stream 388 is sent to the PIPB column.

### EXAMPLE 1

The following example illustrating an embodiment of the present invention is based in part on simulated pilot plant data.

Cumene was contacted with propylene in a circulating, adiabatic alkylator operated at 400 psig and 140° C. inlet with propylene WHSV of 0.42 hr⁻¹ to produce a crude DIPB stream as the alkylator effluent. The alkylation catalyst was MCM-22. The mole ratio of cumene to propylene was 4.0. The weight ratio of cumene feed to effluent re-circulation was 0.1.

The crude DIPB stream was mixed with benzene in a once-through, isothermal transalkylator operated at 400 psig, and 175° C. with crude DIPB WHSV of 0.65 hr⁻¹. The catalyst in the transalkylator was also MCM-22. The weight ratio of benzene to crude DIPB was 3.0. Downstream operations to recover high purity MDIPB and, if desired, PDIPB product were generally as described above in connection with FIG. 2.

The purity of MDIPB and PDIPB products achieved with this process are 99.2 wt. % and 99.6 wt. %, respectively, if distillation is used for their purification. If crystallization is used instead for PDIPB purification, 99.9 wt. % pure PDIPB can be produced.

### EXAMPLE 2

This example illustrating an embodiment of the present invention is based in part on simulated pilot plant data.

Cumene was contacted with propylene in a circulating, adiabatic alkylator operated at 400 psig and 140° C. inlet with propylene WHSV of 0.42 hr⁻¹ to produce a crude DIPB stream as the alkylator effluent. The alkylation catalyst was MCM-22. The mole ratio of aromatics to propylene was 4.0. The weight ratio of cumene feed to effluent re-circulation was 0.1.

The crude DIPB stream was mixed with benzene in a once-through, isothermal transalkylator operated at 400 psig, and 175° C. The catalyst in the transalkylator was also MCM-22. The weight ratio of benzene to crude DIPB was 3.0. The MDIPB/PDIPB product ratio can be varied by changing either the operating conditions of the transalkylator, or the alkylator feed composition, or some combination of these parameters. Downstream operations to recover high purity MDIPB and, if desired, PDIPB product were generally as described above in connection with FIG. 2.

Table 1 below illustrates how varying the operating conditions of the transalkylator or the alkylator feed composition can affect the product ratio of MDIPB to PDIPB in the transalkylator effluent.

TABLE 1

| PIPB conversion in TRA | Benzene/Cumene to ALK | MDIPB/PDIPB |
|---|---|---|
| (%) | wt % | product ratio |
| 41 | 0 | 2:1 |
| 28 | 8 | 2:1 |
| 50 | 0 | 0.86:1 |
| 33 | 5 | 0.86:1 |

### EXAMPLE 3

This example illustrating an embodiment of the present invention is based in part on simulated pilot plant data.

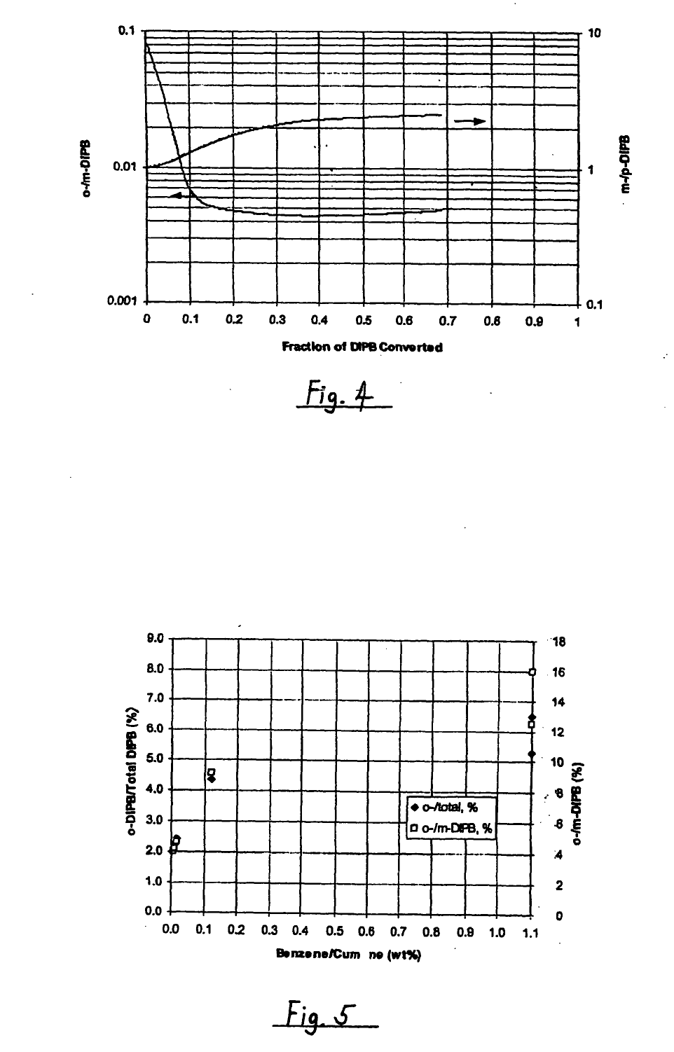

Production of meta-DIPB utilizing a combination of alkylation and transalkylation as described for Examples 1 and 2 above was carried out under varying transalkylation conditions such that the fraction of DIPB conversion varied from about 0 (no conversion) to about 1.0 (complete conversion). At selected fractions of DIPB conversion between 0 and 1, determinations were made of the proportion (by weight or mole ratio—which are identical when comparing isomers) of ortho-DIPB to meta-DIPB and of meta-DIPB to para-DIPB. Those results are plotted in FIG. 4 on a semi-logarithmic scale. Variations in the ortho-isomer to meta-isomer proportions with changes in fraction of DIPB converted (horizontal axis) are plotted using the left vertical axis resulting in the curve identified by the left-pointing arrow. Variations in the meta-isomer to para-isomer proportions with changes in fraction of DIPB converted (horizontal axis) are plotted using the right vertical axis resulting in the curve identified by the right-pointing arrow.

FIG. 4 demonstrates still another surprising and novel advantage of recovering a meta-DIPB-rich stream as transalkylator effluent in accordance with the present invention. FIG. 4 shows that over a wide range of transalkylator operating conditions, ranging from about a 0.2 fraction of DIPB conversion to about a 0.7 fraction of DIPB conversion, the proportion (by weight or mole ratio) of ortho-isomer to meta-isomer remains at very low levels below about 0.005. Because of the relatively close boiling points of ortho-DIPB and meta-DIPB, keeping the proportion of ortho-DIPB to meta-DIPB in the transalkylator effluent to very low levels is critical to ultimately obtaining a high purity meta-DIPB product.

Thus, FIG. 4 demonstrates that, utilizing the methods of the present invention, a high level of conversion of DIPB of up to about 70% is completely compatible with maintaining very low proportions (below 0.005) of ortho-isomer to meta-isomer. At conversion levels above about 80%, it is expected that the o/m-DIPB curve would turn upward reflecting higher proportionate production of ortho-isomer. Such a surprising finding has not previously been reported and is not suggested by any comparable prior art approaches to production of dialkylbenzene compounds.

In addition, FIG. 4 demonstrates that a high conversion of DIPB to about 70% or higher utilizing the methods of the present invention is also completely compatible with obtaining very high, dominant proportions above 2.5/1, or greater than 70%, (by weight or mole ratio) of meta-isomer to para-isomer, while also maintaining the ortho-isomer to meta-isomer proportion at very low levels below about 0.005. Such a surprising finding has not previously been reported and is not suggested by any comparable prior art approaches to production of dialkylbenzene compounds.

### EXAMPLE 4

This example illustrating an embodiment of the present invention is based in part on simulated pilot plant data.

Production of meta-DIPB utilizing a combination of alkylation and transalkylation as described for Examples 1 and 2 above was carried out under conditions of varying compositions of feed to the alkylator with the weight % of benzene/cumene in the alkylator feed varying from about 0.0 (all cumene feed) to about 1.1 wt. % benzene/cumene. At selected wt. % levels of benzene/cumene between 0.0 and 1.1 wt. %, determinations were made of the proportion (by weight or mole ratio) of ortho-DIPB to total DIPB produced and of ortho-DIPB to meta-DIPB produced. Those results are plotted in FIG. 5, with the ortho-DIPB to total DIPB proportion measured along the left vertical axis against variations in the wt. % of benzene/cumene (horizontal scale), and with the ortho-DIPB to meta-DIPB proportion measured along the right vertical axis against variations in the wt. % of benzene/cumene in the alkylator feed.

It has been found advantageous to include at least small amounts of benzene (fresh, or recycled, or a mixture of both) in the alkylator feed to minimize the formation of undesirable heavier aromatic byproducts such as the triisopropylbenzenes (TIPB).

FIG. 5 shows, however, that there are both limits to and efficiency costs for including even very small amounts of benzene in the alkylator feed because of the increased formation of the undesirable ortho-isomer. FIG. 5 illustrates that both the proportion of ortho-DIPB to total DIPB as well as the proportion of ortho-DIPB to meta-DIPB increase consistently and substantially as the wt. % of benzene to cumene in the alkylator feed goes from 0.0 to 1.1. This example therefore demonstrates the surprising and novel discovery as part of the present invention that, in order to maximize meta-isomer production while maintaining very low levels of ortho-isomer, it is desirable to use either an all-cumene feed to the alkylator or at least to limit benzene in the alkylator feed to a benzene/cumene wt. % of about 0.2 or less, preferably about 0.1 or less.

### EXAMPLE 5

This example illustrates typical alkylation stage conditions and compares alkylator effluent compositions utilizing MCM-22 as the alkylation catalyst with two different feed compositions: 5A, a cumene and propylene feed; 5B, a benzene and propylene feed.

5A. Cumene/Propylene Feed to Alkylator

Temperature: 130° C.-190° C. inlet; adiabatic operation; cumene/propylene molar ratio ranges from about 2.5/1 to 8/1; propylene WHSV 0.30-0.60 hr.⁻¹; recirculation/feed ratio about 9-10.

Under the foregoing conditions, the m/o/p-DIPB ratio in the alkylator effluent ranges from about 51 to 59/1.2 to 2.7/39 to 45 respectively.

5B. Benzene/Propylene Feed to Alkylator

Temperature: 130° C. inlet, adiabatic operation; benzene/propylene molar ratio about 4/1; propylene WHSV 0.50 hr.⁻¹; recirculation/feed ratio about 9-10.

Under the foregoing conditions, the m/o/p-DIPB ratio in the alkylator effluent ranges from about 28 to 31/10 to 11/59 to 60 respectively.

This example demonstrates the benefit of utilizing a mixed cumene/propylene feed to the alkylator in order to maximize meta-isomer and minimize ortho-isomer in the alkylator effluent. In accordance with the present invention, however, the alkylator effluent from either 5A. or 5B. may be sent to a downstream meta-isomer enhancement step, as discussed below in Example 6, to ultimately yield an effluent stream which has a predominant proportion (e.g., above 60%) of meta-isomer and a very low proportion (e.g., below 0.5%) of ortho-isomer.

### EXAMPLE 6

This example illustrates typical transalkylator stage conditions and compares transalkylator effluent compositions utilizing MCM-22 as the transalkylation catalyst, and a mixed benzene and crude-PIPB feed, with crude-PIPB originating from an associated alkylation stage, under two different sets of operating conditions, 6A and 6B.

6A. Operating Conditions

Temperature: 170° C.-185° C.; isothermal, once-through operation; benzene/PIPB weight ratio 2-3; PIPB WHSV 0.5-1.25 hr.⁻¹.

Under the foregoing conditions, the m/o/p-DIPB ratio in the transalkylator effluent ranges from about 60 to 69/0.28 to 0.31/30 to 40 respectively.

6B. Operating Conditions

Temperature: 180° C.-185° C.; isothermal, once-through operation; benzene/PIPB weight ratio 3; PIPB WHSV 0.55-0.60 hr.⁻¹.

Under the foregoing conditions, the m/o/p-DIPB ratio in the transalkylator effluent ranges from about 69 to 70/0.31 to 0.33/29 to 31 respectively.

This example demonstrates the effectiveness according to the present invention of utilizing a meta-isomer enhancement step, such as a transalkylation stage with MCM-22 catalyst, in combination with an alkylation stage producing a crude-DIPB stream, to obtain an effluent stream containing a predominant portion above 60% of the meta-isomer while maintaining very low levels well below 0.50%, indeed below 0.35%, of the ortho-isomer. No prior art process of this type produces such high levels of meta-DIPB while maintaining such low levels of ortho-DIPB.

It will be apparent to those skilled in the art that other changes and modifications may be made in the above-described apparatus and methods for producing high purity meta-isomers of dialkylbenzene compounds without departing from the scope of the invention herein, and it is intended that all matter contained in the above description shall be interpreted in an illustrative and not a limiting sense.

We claim:

1. A process for preparing a mixed dialkylbenzene product comprising a predominant proportion above 60 wt, % of the meta-dialkylbenzene isomer and a very low proportion of the ortho-dialkylbenzene isomer from a feed consisting essentially of a suitable starting olefin and a suitable starting aromatic hydrocarbon, which process comprises a liquid-phase alkylation step utilizing an alkylation catalyst selected for enhancement of meta-isomer formation followed by or carried out in combination with a meta-isomer enhancement step utilizing a meta-isomer enhancement catalyst selected for enhancement of meta-isomer formation; and
wherein said dialkylbenzene product comprises at least about 64 wt. % of the meta-dialkylbenzene isomer and less than about 1 wt. % of the ortho-dialkylbenzene isomer.

2. The process according to claim 1 wherein said meta-isomer enhancement catalyst consists essentially of a member selected from the group consisting of zeolite beta, acidic zeolite-based catalysts and mixtures thereof.

3. The process according to claim 1 wherein said alkylation catalyst consists essentially of a member selected from the group consisting of zeolite beta, acidic zeolite-based catalysts and mixtures thereof.

4. The process according to claim 1 wherein both of said alkylation catalyst and said meta-isomer enbancement catalyst consist essentially of the same or different members selected from the group consisting of zeolite beta, acidic zeolite-based catalysts and mixtures thereof.

5. The process according to claims 1-4 wherein the acidic zeolite-based catalyst is MCM-22, MCM-36, MCM-49, MCM-56, ITQ-1, ITQ-2, ITQ-3, SSZ-25 or PSH3.

6. The process according to any of claims 1-5 wherein said meta-isomer enhancement step consists of a transalkylation step.

7. The process according to any of claims 1-5 wherein said meta-isomer enhancement step consists of an isomerization step.

8. The process according to any of claims 1-5 wherein said meta-isomer enhancement step consists of a disproportionation step.

9. The process according to any of claims 1-5 wherein said alkylation step is carried out in combination with a transalkylation step in a combination alkylator/transalkylator.

10. The process according to any of claims 1-5 wherein said meta-isomer enhancement step consists of a combination of at least two steps selected from the group consisting of transalkylation, isomerization and disproportionation.

11. The process according to any of claims 1-5 wherein said starting olefin is propylene, said starting aromatic hydrocarbon consists esssentially of cumene, and said meta-dialkylbenzene isomer is meta-diisopropylbenzene.

12. The process according to any of claims 1-5 wherein said starting olefin is propylene, said starting aromatic hydrocarbon is a mixture of cumene and benzene having a ratio of benzene to cumene of less than about 0.2 wt. %, and said meta-dialkylbenzene isomer is meta-diisopropylbenzene.

13. The process according to claim 1 further comprising the steps of separating a high purity meta-dialkylbenzene isomer product from said mixed dialkylbenzene product, and recycling substantially all of the remaining polyalkylbenzene components of said mixed dialkylbenzene product to the meta-isomer enhancement step to maximize meta-isomer recovery.

14. The process according to claim 1 further comprising the steps of separating a high purity meta-alkylbenzene isomer product from said mixed dialkylbenzene product, separating a high purity para-dialkylbenzene isomer product from the remaining polyalkylbenzene components of said mixed dialkylbenzene product, and recycling substantially all of the still remaining polyalkylbenzene components of said mixed dialkylbenzene product to the meta-isomer enhancement step.

15. The process according to claim 1 wherein said starting olefin is propylene, said starting aromatic hydrocarbon is benzene, and wherein said process fiber comprises the step of separating cumene from the alkylation reactor effluent to produce a crude mixed diisopropylbenzene product before sending the crude mixed diisopropylbenzene product to the meta-isomer enhancement step.

16. The process according to claim 15 further comprising the step of separating a high purity meta-diisopropylbenzene isomer product from said mixed dialkylbenzene product, and recycling substantially all of the remaining polyalkylbenzene components of said mixed dialkylbenzene product to the meta-isomer enhancement step.
